(19) United States
(12) Patent Application Publication
(10) Pub. No.: US 2013/0116274 A1
(43) Pub. Date: May 9, 2013

(54) THERAPEUTIC COMBINATIONS CONTAINING RILUZOLE

(71) Applicant: Rutgers, The State University of New Jersey, New Brunswick, NJ (US)

(72) Inventor: Suzie Chen, Highland Park, NJ (US)

(73) Assignee: RUTGERS, THE STATE UNIVERSITY OF NEW JERSEY, New Brunswick, NJ (US)

(21) Appl. No.: 13/673,590

(22) Filed: Nov. 9, 2012

Related U.S. Application Data
(60) Provisional application No. 61/557,516, filed on Nov. 9, 2011.

(51) Int. Cl. A61K 31/44; A61K 31/428; A61K 31/4375; A61K 45/06

(52) U.S. Cl. CPC A61K 31/44; A61K 45/06; A61K 31/428; A61K 31/4375
USPC 514/293; 514/346

(57) ABSTRACT

Disclosed is a method of treating melanoma in a mammal comprising administering (a) a therapeutically effective amount of an inhibitor of metabotropic glutamate receptor 1 (GRM1); and (b) a therapeutically effective amount of an inhibitor of at least one downstream signaling target of GRM1. Also disclosed are compositions and kits for treating melanoma comprising (a) a therapeutically effective amount of an inhibitor of GRM1; and (b) a therapeutically effective amount of an inhibitor of at least one downstream signaling target of GRM1.

### CROSS-REFERENCE TO RELATED APPLICATIONS

This application claims the benefit of priority under 35 USC §119(e) of U.S. Provisional Application No. 61/557,516, filed on Nov. 9, 2011, the entire disclosure of which is incorporated herein by reference.

### STATEMENT REGARDING FEDERALLY SPONSORED RESEARCH OR DEVELOPMENT

This invention was made with government support under Contract Nos. GM66338, R01CA74077, R01CA124975, R01CA124975-02S1 and 5R25GM055145-16 awarded by National Institutes of Health (NIH), and under Contract No. ES-005022 awarded by the National Institute of Environmental Health Sciences (NIEHS). The US government has certain rights in the invention.

### FIELD OF THE INVENTION

The invention is directed to the field of cancer treatment, specifically melanoma treatment, using a combination of a metabotropic glutamate receptor 1 (GRM1) inhibitor, such as riluzole, with an inhibitor of a downstream signaling target of GRM1.

### BACKGROUND OF THE INVENTION

The incidence of melanoma skin cancer has risen rapidly over the past three decades, and has become a significant health risk in the United States. The standard treatment for early stage melanoma is surgical resection, with over 85% of patients in the early stages of disease experiencing long-term survival. However, when melanoma metastasizes the prognosis is poor, with few patients diagnosed with stage 1V disease surviving past five years.

Standard cytotoxic chemotherapeutic regimens have failed to alter the outcome in patients with advanced disease and only the use of biological therapies based on interleukin-2 (IL-2) demonstrate any effect in extending long-term survival. Previously, traditional melanoma chemotherapy involved the combination of dacarbazine (DTIC), the first drug approved for melanoma treatment which has a 20% response rate as a single agent, with other drugs. Combinations such as the Dartmouth regimen (DTIC, bis-chloroethylnitrosourea (BCNU), cisplatin and tamoxifen) yielded a 40% response rate, or ˜40-62% response rates with the BOLD regimen (bleomycin, vincristine, lomustine, DTIC). However, none of these regimens has demonstrated an increase in survival of stage 1V melanoma patients notwithstanding that they are also associated with increased toxicity compared to DTIC-only therapy. Most recently, the identification of compounds for targeted therapies such as Vemurafenib targeting the BRAFV600E mutation or Ipilimumab targeting the cytotoxic T-lymphocyte antigen-4 (CTLA-4) antigen have shown promise in suppressing tumor growth. Despite these advances, the clinical responses are not durable and relapse is a near certainly spurring the need for new therapies.

Over the past decade, the understanding of the genetic alterations that lead to melanomagenesis and melanoma progression has advanced. Key signaling pathways involved in the pathogenesis and progression of melanoma, including the AKT, MAPK, PI3K/AKT, Wnt, JNK, TGF-β, and NFκB, suggest a molecularly complex and heterogeneous disease.

Metabotropic glutamate receptors (GRMs) are members of the seven-transmembrane domain G-protein-coupled receptor (GPCR) family. GRMs are divided into three groups based on sequence homology, agonist selectivity and effecter coupling, with all GRMs having glutamate as their natural ligand. GRM1 and GRM5 comprise Group I GRMs and are mainly involved in excitatory responses induced by strong presynaptic stimulation. Group I GRMs are coupled to a Gαq-like protein and stimulate phospholipase C beta (PLCβ). In melanoma cells, GRM1 stimulation results in the activation of PLCβ, which in turn converts phosphatidylinositol to two second messengers, inositol triphosphate (IP₃) and diacylglycerol (DAG). DAG activates protein kinase C (PKC), which could stimulate both MAPK and phosphoinositide 3-kinase (PI3K)/AKT pathways. Activation of these two major signaling cascades is central for transformed cell survival, migration, invasion, epithelial-mesenchymal transition (EMT), and angiogenesis.

The PI3K/AKT pathway plays a fundamental role in apoptosis and tumorigenesis. This pathway is a known regulator of cell differentiation, proliferation, division, survival and tumorigenesis. Numerous components of this pathway are deregulated or mutated in various cancers including melanoma. Inactivation or loss of the tumor suppressor phosphatase and tensin homologue (PTEN) has been reported in 30-50% of melanomas, while genetic amplification of AKT3 has been reported in 43-60% of melanomas resulting in increased PI3K/AKT signaling. AKT2 has also been reported to be involved in melanoma and especially those that express GRM1, where it has been shown to be a downstream target of the receptor.

The US Food and Drug Administration (FDA)-approved drug, riluzole, is a member of the benzothiazole class of compounds which acts as an inhibitor of glutamate release for the treatment of amyotrophic lateral sclerosis (ALS). The ability of riluzole to block the release of the ligand (glutamate) for GRM1 allows it to act functionally as a putative antagonist and interfere with intracellular events that follow stimulation of this receptor. Riluzole possesses a known, low toxicity profile. To date, the reported modes of actions of riluzole in humans include inhibition of glutamate release, inactivation of voltage-dependent Na⁺ channels, and interference with G-protein dependent signaling. In melanoma cells expressing GRM1, riluzole has been shown to inhibit cell proliferation in vitro and in vivo, as well as migration and invasion. Recently, a Phase 0 clinical trial of riluzole in patients with advanced melanoma was conducted with 34% of patients given riluzole showing measurable clinical responses. Some tumors decreased in size by over 90% and exhibited suppression of MAPK and PI3K/AKT signaling pathways in post-treatment tumor samples. A recently completed Phase II trial showed no RECIST criteria responses; however, 42% of the patients exhibited stable disease suggesting that riluzole has overall modest anti-tumor activity.

### BRIEF SUMMARY OF THE INVENTION

In accordance with the present invention, it has been discovered that the combination of a GRM1 inhibitor with an inhibitor of a downstream signaling target of GRM1 provides a superior treatment for melanoma, both in vitro and in vivo.

One aspect of the invention is directed to a method of treating melanoma in a mammal comprising administering (a) a therapeutically effective amount of an inhibitor of metabotropic glutamate receptor 1 (GRM1); and (b) a therapeutically effective amount of an inhibitor of at least one downstream signaling target of GRM1. Preferably, the mammal is a human being. The inhibitor of GRM1 is referred to herein as “inhibitor (a)”. The inhibitor of at least one downstream signaling target of GRM1 is referred to herein as “inhibitor (b)”.

In one embodiment of the method of treating melanoma, the inhibitor of GRM1 comprises riluzole.

In another embodiment of the method of treating melanoma, the downstream signaling target of GRM1 is selected from the group consisting of AKT, MAPK, PI3K/AKT and a combination of two or more thereof. In one embodiment, the inhibitor (b) is selected from the group consisting of MK2206, sorafenib and a mixture thereof. In a preferred embodiment, the inhibitor (b) comprises MK2206. In another preferred embodiment, the inhibitor (b) comprises sorafenib. In particularly preferred embodiments of the invention, the inhibitor (a) comprises riluzole and the inhibitor (b) comprises MK2206 or sorafenib.

In one embodiment of the method of treating melanoma, inhibitors (a) and (b) are co-administered. Inhibitors (a) and (b) can be co-administered as a combination. In one embodiment, the combination can be a unitary formulation; that is, both inhibitors are co-formulated together.

In one embodiment of the method of treating melanoma, inhibitors (a) and (b) can be administered separately, with or without a time delay. Preferably inhibitors (a) and (b) are formulated compositions.

Another aspect of the invention is directed to a composition for treating melanoma comprising (a) a therapeutically effective amount of an inhibitor of metabotropic glutamate receptor 1 (GRM1); and (b) a therapeutically effective amount of an inhibitor of at least one downstream signaling target of GRM1.

In one embodiment of the composition, the inhibitor of GRM1 comprises riluzole.

In another embodiment of the composition, the downstream signaling target of GRM1 is selected from the group consisting of AKT, MAPK, PI3K/AKT and a combination of two or more thereof.

In another embodiment of the composition, the inhibitor (b) is selected from the group consisting of MK2206, sorafenib and a mixture thereof. In one preferred embodiment, the inhibitor (b) comprises MK2206. In another preferred embodiment, the inhibitor (b) comprises sorafenib.

In a preferred embodiment of the composition, the inhibitor (a) comprises riluzole and the inhibitor (b) comprises MK2206. In another preferred embodiment of the composition, the inhibitor (a) comprises riluzole and the inhibitor (b) comprises sorafenib.

Another aspect of the invention is directed to a kit for the treatment of melanoma comprising a container comprising (a) an inhibitor of GRM1 and a container comprising (b) an inhibitor of at least one downstream signaling target of GRM1.

In one embodiment of the kit, the inhibitor of GRM1 comprises riluzole.

In another embodiment of the kit, the downstream signaling target of GRM1 is selected from the group consisting of AKT, MAPK, PI3K/AKT and a combination of two or more thereof.

In another embodiment of the kit, the inhibitor (b) is selected from the group consisting of MK2206, sorafenib and a mixture thereof. In one preferred embodiment, the inhibitor (b) comprises MK2206. In another preferred embodiment, the inhibitor (b) comprises sorafenib.

In a preferred embodiment of the kit, the inhibitor (a) comprises riluzole and the inhibitor (b) comprises MK2206. In another preferred embodiment of the kit, the inhibitor (a) comprises riluzole and the inhibitor (b) comprises sorafenib.

### BRIEF DESCRIPTION OF THE DRAWINGS

FIG. 1 illustrates the treatment of melanoma cells with three different inhibitors. MTT assays of C8161 (A, B-RAF and N-RAS wild type), UACC903 (B, B-RAF^{V600E}) and 1205Lu, (C, B-RAF^{V600E}) melanoma cells treated with different concentrations of riluzole, sorafenib, PLX4720 or the combination of the compounds at half of the concentration of each one alone. NT—No treatment; DMSO—Vehicle only treatment: riluzole (Ril); sorafenib (Sor); PLX4720 (PLX).

FIG. 2 illustrates the inhibition of anchorage independent growth with riluzole and sorafenib alone and in combination. Colony assays were performed with C8161 (B-RAF and N-RAS wild type), UACC903, 1205Lu (B-RAF^{V600E}), and SKMEL2 (N-RAS^{Q61R}) GRM1 positive human melanoma cell lines. Colony number and size were determined using ImageJ software. Results are the mean of 3 independent experiments.

FIG. 3 illustrates the suppression of human melanoma xenografts growth. Xenografts of A: C8161 (B-RAF and N-RAS wild type), B: UACC903 (B-RAF^{V600E}) and C: 1205Lu (B-RAF^{V600E}). The groups were no treatment (NT), vehicle (Veh, DMSO), riluzole (10 mg/kg), sorafenib (24 mg/kg) or the combination of riluzole (5 mg/kg) and sorafenib (12 mg/kg). Tumor volume (mm³) was an average of 12 mice per group ±S.D. * p<0.01 when riluzole- or sorafenib-treated UACC903 samples were compared to NT or Veh controls at day 18. ** p<0.001 when riluzole-treated C8161 or the combination of riluzole plus sorafenib-treated C8161, UACC903 or 1205Lu samples were compared to NT or Veh controls. D: Xenograft experiments of UACC903 treated with either riluzole (10 mg/kg), PLX4720 (20 mg/kg) or a combination of riluzole (5 mg/kg) and PLX4720 (10 mg/kg), * p<0.05 in comparison to NT or Veh.

FIG. 4 illustrates MTT cell proliferation and viability assays examining effects of various concentrations of riluzole in C8161, UACC903 and SKMEL 28 human melanoma cell lines. NT—No treatment, Vehicle (DMSO), Ril—riluzole.

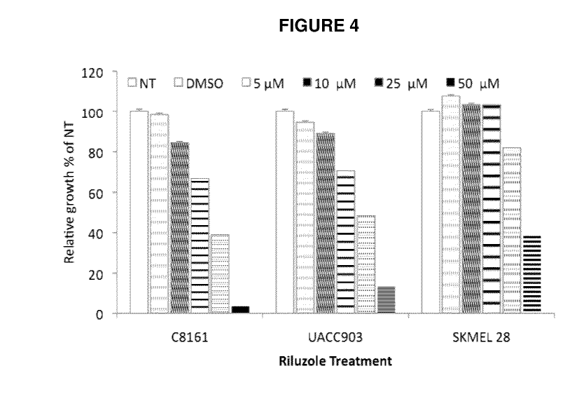

FIG. 5 illustrates A. MTT cell proliferation and viability assays examining the effects of various concentrations of MK2206 in C8161, UACC903 and SKMEL 28 human melanoma cells. B. MTT cell proliferation and viability assays examining the effects of the combination of riluzole and MK2206 on the three different human melanoma cell lines. NT—No treatment, Vehicle (DMSO).

FIG. 6 illustrates in vivo C8161 human melanoma xenografts in nude athymic mice, and treatment with riluzole alone (RIL, 10 mg/kg), MK2206 alone (MK, 30 mg/kg) or a combination of riluzole (5 mg/kg) and MK2206 (15 mg/kg).

### DETAILED DESCRIPTION OF PREFERRED EMBODIMENTS

The present invention is directed to melanoma treatment combining inhibition of GRM1, for example using riluzole, and inhibition of a downstream signaling target of GRM1, such as AKT, MAPK or PI3K/AKT.

One embodiment of the present invention is directed to melanoma treatment comprising administering to a mammal in need of such treatment:
(a) a therapeutically effective amount of an inhibitor of metabotropic glutamate receptor 1 (GRM1); and
(b) a therapeutically effective amount of an inhibitor of at least one downstream signaling target of GRM1.

Preferably, the mammal is a human being.

The term “therapeutically effective amount”, as used herein, refers to a level which is commonly known in the art and recognized and utilized by the medical community. Such an amount will vary depending on the particular agent(s) administered, the size and/or condition of the individual receiving treatment or other medical factors determined by the administering physician. A therapeutically effective amount is a nontoxic but sufficient amount of an active agent to provide the desired therapeutic effect.

Preferably the inhibitor of GRM1 comprises riluzole. More preferably the inhibitor of GRM1 consists essentially of riluzole. Most preferably the inhibitor of GRM1 is riluzole.

Preferably, the downstream signaling target of GRM1 is selected from the group consisting of AKT, MAPK, PI3K/AKT and a combination of two or more thereof.

In one embodiment, the inhibitor of the downstream signaling target of GRM1 is selected from the group consisting of MK2206, sorafenib and a mixture thereof. In a particular embodiment, the downstream signaling inhibitor comprises sorafenib. Preferably the inhibitor consists essentially of sorafenib. More preferably the inhibitor is sorafenib. Sorafenib (formula (I)) is a small-molecule multi-kinase inhibitor which is a MAPK pathway inhibitor, and has been shown to inhibit RAF signaling. The toxicity profile of sorafenib is known in vivo.

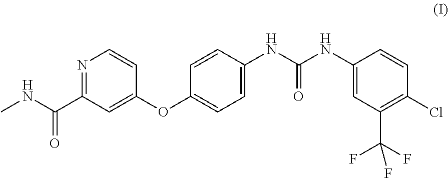 [SMILES: CNC(=O)c1cc(Oc2ccc(NC(=O)Nc3ccc(Cl)c(C(F)(F)F)c3)cc2)ccn1]

In another particular embodiment, the downstream signaling inhibitor comprises MK2206, an AKT inhibitor. Preferably the inhibitor consists essentially of MK2206. More preferably the inhibitor is MK2206. The chemical structure of MK2206, CAS no. 1032350-13-2, is shown in formula (II):

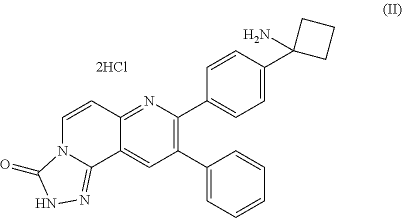 [SMILES: NC1(c2ccc(-c3nc4ccn5c(=O)[nH]nc5c4cc3-c3ccccc3)cc2)CCC1]

In a preferred method, the inhibitor of metabotropic glutamate receptor 1 comprises riluzole and the inhibitor of the downstream signaling target comprises MK2206. More preferred is the method wherein the inhibitor of GRM1 consists essentially of riluzole and the inhibitor of the downstream signaling target consists essentially of MK2206. In a particularly preferred method, the inhibitor of GRM1 is riluzole and the inhibitor of the downstream signaling target is MK2206. Also, the inhibitor of GRM1 of a preferred method can also comprise riluzole, while the inhibitor of the downstream signaling target consists essentially of MK2206 or is MK2206. Further, the inhibitor of GRM1 of a preferred method can consist essentially of riluzole or be riluzole, while the inhibitor of the downstream signaling target comprises MK2206.

In another preferred method, the inhibitor of metabotropic glutamate receptor 1 comprises riluzole and the inhibitor of the downstream signaling target comprises sorafenib. More preferred is the method wherein the inhibitor of GRM1 consists essentially of riluzole and the inhibitor of the downstream signaling target consists essentially of sorafenib. In a particularly preferred method, the inhibitor of GRM1 is riluzole and the inhibitor of the downstream signaling target is sorafenib. Also, the inhibitor of GRM1 of a preferred method can also comprise riluzole, while the inhibitor of the downstream signaling target consists essentially of sorafenib or is sorafenib. Further, the inhibitor of GRM1 of a preferred method can consist essentially of riluzole or be riluzole, while the inhibitor of the downstream signaling target comprises sorafenib.

Inhibitors (a) and (b) can be administered as the individual active ingredients (ai's), or as formulated products. Preferably the administration of the ai's is in formulated form, with the formulations comprising at least one pharmaceutically acceptable carrier or additive. In a method of the invention inhibitors (a) and (b) can be co-administered. Inhibitors (a) and (b) can be co-administered as a combination, such as a unitary formulation, or as separate formulations. When inhibitors (a) and (b) are administered separately, they may be administered in any order, and the time delay between administration of the individual formulations can range from about 5 minutes to about 24 hours. Preferably the time delay is in the range of about 1 to about 12 hours.

Another embodiment of the invention is directed to a composition for treating melanoma comprising:
(a) a therapeutically effective amount of an inhibitor of metabotropic glutamate receptor 1 (GRM1); and
(b) a therapeutically effective amount of an inhibitor of at least one downstream signaling target of GRM1.

Optionally, the composition may further comprise one or more pharmaceutically acceptable carriers and/or additives.

Preferably the inhibitor of GRM1 comprises riluzole. More preferably the inhibitor of GRM1 consists essentially of riluzole. Most preferably the inhibitor of GRM1 is riluzole.

Preferably, the downstream signaling target of GRM1 is selected from the group consisting of AKT, MAPK, PI3K/AKT and a combination of two or more thereof.

In one embodiment, the inhibitor of the downstream signaling target of GRM1 is selected from the group consisting of MK2206, sorafenib and a mixture thereof. In a particular embodiment the downstream signaling inhibitor comprises MK2206. Preferably the inhibitor consists essentially of MK2206. More preferably the inhibitor is MK2206. In another particular embodiment the downstream signaling inhibitor comprises sorafenib. Preferably the inhibitor consists essentially of sorafenib. More preferably the inhibitor is sorafenib.

In a preferred composition, the inhibitor of metabotropic glutamate receptor 1 comprises riluzole, and the inhibitor of the downstream signaling target comprises MK2206. More preferred is the composition wherein the inhibitor of GRM1 consists essentially of riluzole and the inhibitor of the downstream signaling target consists essentially of MK2206. In a particularly preferred composition, the inhibitor of GRM1 is riluzole and the inhibitor of the downstream signaling target is MK2206. A preferred composition can also comprise riluzole, while the downstream signaling target consists essentially of MK2206 or is MK2206. Further, another preferred composition can consist essentially of riluzole or be riluzole, while the downstream signaling target comprises MK2206.

In another preferred composition, the inhibitor of metabotropic glutamate receptor 1 comprises riluzole, and the inhibitor of the downstream signaling target comprises sorafenib. More preferred is the composition wherein the inhibitor of GRM1 consists essentially of riluzole and the inhibitor of the downstream signaling target consists essentially of sorafenib. In a particularly preferred composition, the inhibitor of GRM1 is riluzole and the inhibitor of the downstream signaling target is sorafenib. A preferred composition can also comprise riluzole, while the downstream signaling target consists essentially of sorafenib or is sorafenib. Further, another preferred composition can consist essentially of riluzole or be riluzole, while the downstream signaling target comprises sorafenib.

In one embodiment of the present invention, the composition comprising the inhibitor is formulated in accordance with standard procedure as a pharmaceutical formulation adapted for delivered administration to human beings and other mammals. Typically, formulations for intravenous administration are solutions in sterile isotonic aqueous buffer.

Where necessary, the formulation may also include a solubilizing agent and a local anesthetic to ameliorate any pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the formulation is to be administered by infusion, it can be dispensed with an infusion bottle containing sterile pharmaceutical grade water or saline. Where the formulation is administered by injection, an ampule of sterile water for injection or saline can be provided so that the ingredients may be mixed prior to administration.

In cases other than intravenous administration, the formulation can contain minor amounts of wetting or emulsifying agents or pH-buffering agents. The formulation can be a liquid solution, suspension, emulsion, gel, polymer, or sustained-release formulation. The formulation can further include traditional binders and carriers, as would be known in the art. Formulations can include standard carriers such as pharmaceutical grades of mannitol, lactose, starch, magnesium stearate, sodium saccharide, cellulose, magnesium carbonate, etc., inert carriers having well established functionality in the manufacture of pharmaceuticals. Various delivery systems are known and can be used to administer a therapeutic of the present invention including encapsulation in liposomes, microparticles, microcapsules, and the like. Thus, in one embodiment, the inhibitors of the invention are added directly, or can be complexed with cationic lipids, packaged within liposomes, packaged within viral vectors, or otherwise delivered to target cells or tissues.

The compositions may be administered by any route that delivers an effective dosage to the desired site of action, with acceptable (preferably minimal) side-effects. Numerous routes of administration of agents are known, for example, oral, rectal, transmucosal, or intestinal administration; parenteral delivery, including intramuscular, subcutaneous, intramedullary injections, as well as intrathecal, intraperitoneal, intranasal, cutaneous or intradermal injections; inhalation, and topical application.

Therapeutic dosing is achieved by monitoring therapeutic benefit and monitoring to avoid side-effects. Preferred dosage provides a maximum localized therapeutic benefit with minimum local or systemic side-effects. Suitable human dosage ranges for the inhibitors can be extrapolated from these dosages or from similar studies in appropriate animal models. Dosages can then be adjusted as necessary by the clinician to provide maximal therapeutic benefit for human subjects.

For example, a therapeutically effective dosage of riluzole may be from 50 to 200 mg/day. A therapeutically effective dosage of sorafenib may be from 50 to 800 mg/day. A therapeutically effective dosage of MK2206 may be from 1 to 30 mg/day.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

The composition may further contain other agents which either enhance the activity of the inhibitor or other active ingredient or complement its activity or use in treatment. Such additional factors and/or agents may be included in the pharmaceutical composition to produce a synergistic effect with protein or other active ingredient, or to minimize side effects.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

Another aspect of the invention is directed to a kit for the treatment of melanoma comprising a container comprising (a) an inhibitor of GRM1 and a container comprising (b) an inhibitor of at least one downstream signaling target of GRM1. Optionally, the kit may further comprise one or more pharmaceutically acceptable carriers and/or additives.

In one embodiment of the kit, the inhibitor of GRM1 comprises riluzole.

In another embodiment of the kit, the downstream signaling target of GRM1 is selected from the group consisting of AKT, MAPK, PI3K/AKT and a combination of two or more thereof.

In another embodiment of the kit, the inhibitor (b) is selected from the group consisting of MK2206, sorafenib and a mixture thereof. In one preferred embodiment, the inhibitor (b) comprises MK2206. In another preferred embodiment, the inhibitor (b) comprises sorafenib.

In a preferred embodiment of the kit, the inhibitor (a) comprises riluzole and the inhibitor (b) comprises MK2206. In another preferred embodiment of the kit, the inhibitor (a) comprises riluzole and the inhibitor (b) comprises sorafenib.

Without wishing to be bound by any theory, it is believed that atypical activation of GRM1 in melanocytes contributes to oncogenesis leading to melanoma development by providing proliferative and survival signals. Moreover, it is believed that interference with GRM1 expression or function, by depletion of its ligand and/or perturbation of downstream effector signaling, promotes tumor cell death, thereby providing a novel strategy for the treatment of melanoma. Riluzole in combination with inhibitors of downstream targets such as MAPK and AKT is effective in suppressing melanoma cell growth both in vitro and in vivo. The AKT inhibitor MK2206 possesses distinct suppressive activities toward downstream AKT pathway(s). This combination shows suppression of melanoma cells in vitro and suppresses melanoma in xenograft models in vivo.

All references cited herein are incorporated by reference herein in their entireties.

### EXAMPLES

Materials and Methods

Antibodies and Reagents

Antibodies against activated Caspase 3, Ki67, PARP, phospho- and total ERK, cleaved PARP, and Mcl-1 were obtained from Cell Signaling (Danvers, Mass.); antibody for a-tubulin, MTT cell viability assay solution 1 (3-(4,5-Dimethylthiazol-2-yl)-2,5-diphenyltetrazolium bromide), iodonitrotetrazolium chloride, riluzole was obtained from Sigma (St. Louis, Mo.); sorafenib (LC labs, Woburn, Mass.).

Cell Lines

UACC903 and UACC930 cells were provided by Dr. Jeffery Trent (The Translational Genomics Research Center, Phoenix, Ariz.) and 1205Lu cells were provided by Dr. Meenhard Herlyn (Wistar Institute, Philadelphia, Pa.). C8161 cells were provided by Dr. Mary Hendrix (Children's Memorial Research Center, Chicago, Ill.). SKMEL2, SKMEL 187 and A2058 cells were purchased from ATCC. The cells were maintained in RPM′ plus 10% FBS. Human epidermal melanocytes (HEM) were maintained in medium 254 (Invitrogen) supplemented with human melanocyte growth supplement (Invitrogen). Human epithelial kidney cells (HEK) were maintained in DMEM plus 10% fetal bovine serum (FBS).

MTT Assays, Cell Cycle Analysis and Glutamate Release

MTT cell viability assays were performed as known in the art. C8161, UACC903, 1205Lu, SKMEL 187 and A2058 melanoma cell lines were used in the MTT assays. Each cell line was cultured in 96 well plates (10³ cells/well) with the following conditions: no treatment, vehicle alone (DMSO), riluzole (1 μM, 5 μM, 10 μM, 25 μM, or 50 μM), sorafenib (1 μM, 3 μM, 5 μM, or 7 μM), the combination of riluzole (10 μM) and sorafenib (5 μM); MK2206 (3 μM, 5 μM or 10 μM) or the combination of riluzole (5 μM or 10 μM) with MK2206 (5 μM or 10 μM). Viable cells were measured every day for 4 or 7 days. Absorbance was determined using a Tecan plate reader (Infinite 200 Tecan USA, Durham, N.C.) at 570 nm everyday for up to 7 days. For cell cycle analysis, UACC903, 1205Lu, and A2058 melanoma cell lines were used. Cell cycle analysis was performed at 24 and 48 hours of incubation of the cell lines in monolayer culture with no treatment, vehicle alone (DMSO), or 10 μM riluzole. Cells were harvested at each time point and examined using propidium iodide followed by flow cytometry performed by the Flow Cytometry Facility Core at Rutgers University. Amplex Red Glutamic Acid/Glutamate Oxidase assay kit (Invitrogen) was used to measure levels of glutamate.

Three Dimensional Anchorage Independent Assays

Three-dimensional colony assays were performed using C8161, UACC903, SKMEL2 and 1205Lu human melanoma cell lines in the presence of vehicle (DMSO), riluzole (10 μM), sorafenib (5 μM), or the combination of riluzole (10 μM) and sorafenib (5 μM). The cells were suspended in 0.35% agar in RPMI supplemented with 10% FBS and plated on a layer of 0.75% agar in the same medium in 12-well culture plates (1−1.5×10⁴ cells/well). Vehicle, riluzole alone, sorafenib alone, or riluzole plus sorafenib, were added in the agar underlay, as well as to the cells suspended in agar on day 1. Every other day, the vehicle, or drug(s) was again added with 250 μl of complete medium. After 14 days, the colonies were stained with iodonitrotetrazolium chloride (Sigma) and photographed. The numbers of colonies were counted using Image J software. Quantitation was performed by comparing the total number of colonies from three representative photomicrographs from each experiment. The histograms represent the average of three independent experiments.

Western Immunoblots

Protein lysates were prepared as commonly known in the art. Briefly, media was removed and cells were washed once with ice-cold phosphate buffered saline (PBS). After removal of PBS, the extraction buffer was added directly to the plates and cells were collected with a cell scraper. Cells were incubated on ice for 20 minutes. Cell debris was removed by centrifugation at 25,000×g at 4° C. for 20 minutes and supernatant taken for Western immunoblot analysis. Western Blotting was carried out with anti-PARP, anti-cleaved PARP, anti-phospho ERK, anti-total ERK, anti-a-tubulin antibodies, anti AKT and anti-Phospho AKT-S473.

Xenografts in Immunodeficient Nude Mice

The Institutional Review Board approved all animal studies for the Animal Care and Facilities Committee of Rutgers University. Nude mice were purchased from Taconic (Hudson, N.Y.). Cells were injected into 2 dorsal sites of each mouse at 10⁶ cells per site. Tumor size was measured twice a week with a Vernier caliper and calculated as described. Once tumor volumes reached 6-10 mm³, mice were divided into no treatment and treatment groups. The treatment groups received either vehicle (DMSO), riluzole (10 mg/kg) daily, sorafenib (24 mg/kg) daily, or the combination of riluzole (5 mg/kg) and sorafenib (12 mg/kg) daily; riluzole (10 mg/kg) daily, MK2206 (30 mg/kg) every three days, or the combination of riluzole (5 mg/kg) and MK2206 (15 mg/kg) daily by oral gavage. The experiments were terminated when the xenografts in the no treatment group reached the maximum permitted size.

Immunohistochemistry

Tissue Analytical Services at the Cancer Institute of New Jersey performed immunohistochemical staining on excised tumor xenografts to detect changes in the number of apoptotic and proliferating cells (activated Caspase 3 and Ki-67, respectively). The number of stained cells was quantified with a Digital Aperio ScanScopeGL system and ImageScope software (v 10.1.3.2028) (Aperio Technologies Inc., Vista, Calif.) according to the manufacturer's protocol.

Results

The oncogenic transformation of various cell types by ectopic expression of GPCRs is characterized by the development of autocrine and paracrine loops that enhance cellular proliferation. Three melanoma cell lines (UACC903, 1205Lu and A2058) containing the activating B-RAF^{V600E} mutation exhibited elevated levels of extra-cellular glutamate similar to that previously described for wild type B-RAF melanoma cells, C8161 and WM239A compared to cells that do not express the receptor or cells that contain a truncated, non-functioning GRM1 receptor, UACC930 melanoma cells. MTT cell viability assays were performed to rule out the possibility that the increase in glutamate observed was not attributable to the cell lysis, thereby establishing that the cells themselves must be excreting glutamate into their surroundings in an attempt to establish autocrine activity. The effects of the glutamate-release inhibitor, riluzole, on the growth of human melanoma cells in monolayer culture were assessed. Standard MTT assays were performed using four GRM1-expressing melanoma cell lines expressing wild type forms of B-RAF and N-RAS (C8161 and SKMEL 187) or B-RAF^{V600E} mutation (UACC903, 1205Lu, and A2058). It was found that riluzole at a concentration of 25 μM or 50 μM significantly decreased the number of viable cells as compared to no treatment, or vehicle (DMSO) treated cells. Melanoma cells harboring a wild type B-RAF (C8161 and SKMEL 187) were found to be much more sensitive to riluzole than those that contained a mutant copy of B-RAF (1205Lu, UACC903). This is supported by findings that indicated that since both GRM1 and B-RAF^{V600E} stimulate MAPK signaling, abolishing only one of these key signaling pathways in human melanoma leading to metastasis, e.g. abolishing GRM1 signaling alone in cells that bear B-RAF^{V600E}, would not abolish over-activated MAPK.

The cell cycle profiles of riluzole-treated (10 μM) UACC903, 1205Lu, and A2058 melanoma cells were obtained to assess the effects that it had on cell cycle progression over time. All three cell lines yielded very similar results. At 24 hours post-treatment about half of the cells were found to accumulate in the G2/M phase. By 48 hours there was a 10-to-20-fold shift of the cell population to the subG1 phase of the cycle, indicative of apoptotic cell response. This apoptotic response was confirmed by an increase in the cleaved form of PARP by Western analysis. Control samples showed negligible amounts of cleaved PARP at 24 and 48 hours. These results demonstrate a G2/M cell cycle arrest followed by apoptotic shift in GRM1-expressing human melanoma cell lines harboring wild type B-RAF and N-RAS (C8161) or mutated N-RAS (WM239A) in the presence of riluzole, indicating that depletion of the ligand (glutamate) to the receptor, GRM1, by riluzole induces cell cycle arrest and promotes apoptosis in GRM1-positive melanoma cells regardless of B-RAF genotype. To confirm this observation in vivo, xenograft experiments were performed using single agent riluzole. Briefly, UACC903 cells were injected into the dorsal flanks of nude mice. Tumors were allowed to grow to approximately 6-10 mm³ and mice were divided into groups to obtain relatively constant tumor volumes between each group (12 mice per group). Animals were treated daily with riluzole or vehicle (DMSO) by oral gavage. At day 18, there was a substantial difference between the tumor sizes of riluzole-treated animals compared to controls. Though riluzole on its own appears effective in inhibiting proliferation and inducing apoptosis in melanoma cells harboring activating B-RAF mutations in vivo, it is less effective at doing so than in melanoma xenografts harboring wild type B-RAF. Clinically, these observations suggest it is likely that administration of single agent riluzole will not be as effective in patients whose melanomas contain a mutated form of B-RAF.

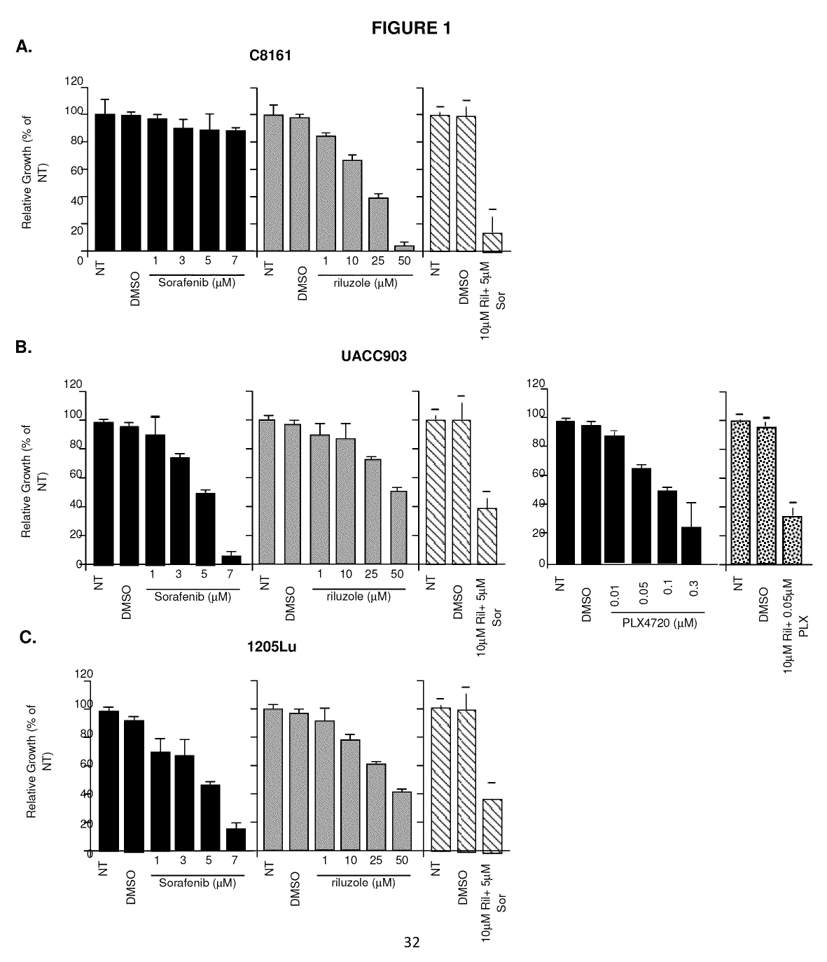

Potential combinatorial therapies that would include riluzole as one of the components to treat heterogeneous tumor populations were investigated, in an attempt to slow the progression of this disease. Sorafenib, a multi-kinase inhibitor which has been shown to inhibit RAF signaling, and whose toxicity profile is known in vivo, and PLX4720, a specific small molecule inhibitor for B-RAF^{V600E}, were assessed. Three GRM1-expressing human melanoma cell lines (C8161, UACC903 and 1205Lu) were treated with riluzole, sorafenib, or a combination of both riluzole and sorafenib for seven days, and cell proliferation and viability were assessed using MTT assays (FIG. 1). In the presence of riluzole alone, the C8161 cell line showed the greatest reduction in the number of viable cells (FIG. 1A). UACC903 and 1205Lu are also positive for GRM1 expression and harbor a mutated B-RAF (V600E). These cell lines were not as sensitive to riluzole (FIGS. 1B and 1C). In the presence of sorafenib, the opposite responses were observed; UACC903 and 1205Lu displayed a substantial decrease in the number of viable cells in comparison to C8161 cells. A combination of 10 μM riluzole with 5 μM sorafenib led to a synergistic, inhibitory effect on the proliferation C8161 cells (FIG. 1A), and an additive, inhibitory effect on UACC903 and 1205Lu cells (FIGS. 1B and 1C). To assess if combining PLX4720 with riluzole would also yield the additive effect observed with sorafenib, UACC903 and C8161 cells were treated with riluzole, PLX4720 or the combination of both. The IC₅₀ for PLX4720 in UACC903 cells was determined to be 0.1 μM (FIG. 1B). UACC903 cells treated with a combination of 10 uM riluzole and half the IC₅₀, 0.05 uM PLX4720 exhibited additive inhibitory activity when compared to either single agent alone (FIG. 1B). Wild type B-RAF, GRM1 positive C8161 cells show only slight inhibition in cell proliferation with higher concentrations of PLX4720 (10 uM) and no increase in efficacy when combined with riluzole.

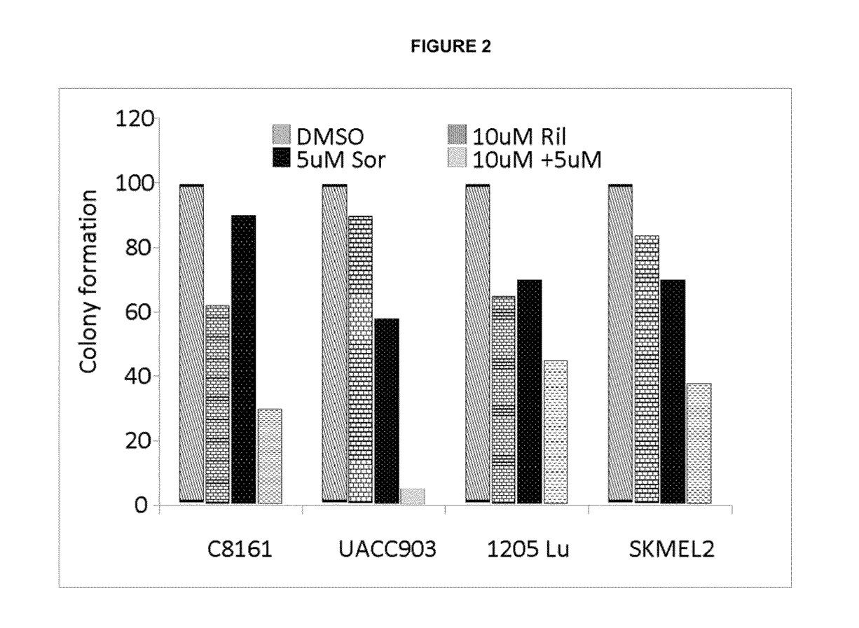

Three-dimensional, anchorage-independence assays were performed using four GRM1-positive melanoma cell lines: C8161 (wild type B-RAF and N-RAS), UACC903, 1205Lu (mutated B-RAF^{V600E}), and SKMEL2 (mutated N-RAS^{Q61R}). In C8161 cells, it was found that riluzole at 10 μM led to a 40% decrease in colony formation while sorafenib alone had little effect (FIG. 2). However, the combination of riluzole and sorafenib had a substantial consequence resulting in a 70% decrease in colony formation (FIG. 2). In UACC903 cells, riluzole alone had very little inhibitory activity while treatment with sorafenib resulted in a 45% reduction in the number of colonies (FIG. 2). Furthermore, the combination of riluzole and sorafenib led to a drastic 90% decrease in the number of colonies in UACC903 (FIG. 2). In 1205Lu cells, riluzole or sorafenib alone yielded a 30% reduction in colony formation while the combination of both resulted in a 55% decrease in the number of colonies (FIG. 2). In SKMEL2, riluzole alone had a modest effect, decreasing colony formation by 18% while sorafenib was more efficacious at decreasing colony formation. The combination treatment yielded a 62% decrease compared to the control group (FIG. 2). These observations indicate that a combination of riluzole and sorafenib would inhibit tumor cell proliferation more effectively than either agent alone, regardless of the presence or absence of activating mutations in B-RAF or N-RAS in the cells.

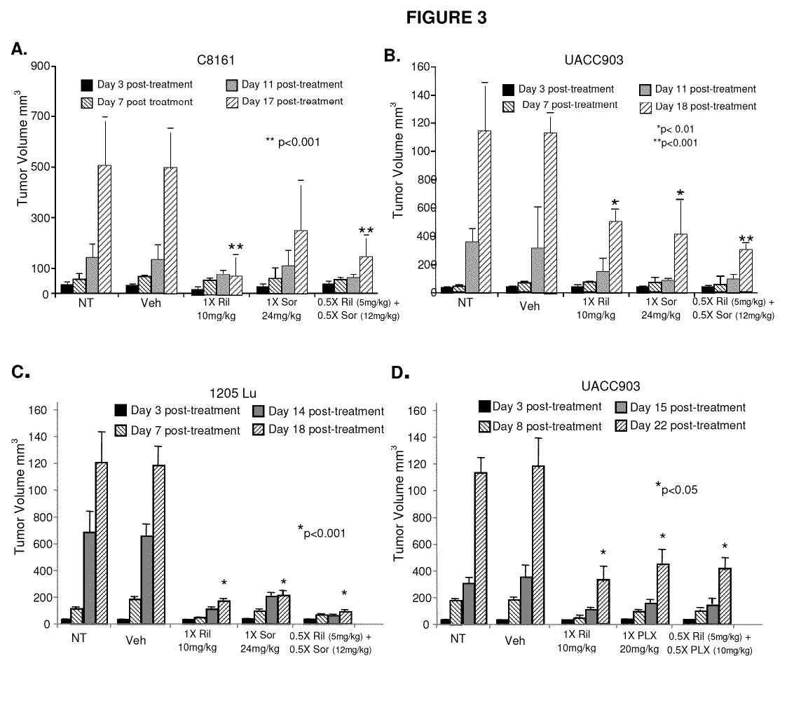

Combinatorial in vivo experiments using C8161, UACC903 and 1205Lu xenografts were performed. In the xenograft studies, all of the cell lines examined express GRM1 but differ in B-RAF genotype, with C8161 being wild type and UACC903 and 1205Lu containing the activating mutation. In C8161 xenografts, there was a significant decrease in the tumor volumes in animals treated with riluzole alone. Administration of sorafenib on its own did not yield a significant decrease in tumor size and the combination of riluzole with sorafenib at half the dose used in either one alone yielded a considerable reduction in tumor volume (FIG. 3A). In the human melanoma cell lines with mutated B-RAF, UACC903 and 1205Lu, differential responses were detected. UACC903 xenografts demonstrated very similar, statistically relevant responses with riluzole or sorafenib alone (FIG. 3B). The combination of riluzole and sorafenib yielded a higher reduction in tumor volume than either compound alone (FIG. 3B).

1205Lu xenografts were found to be more sensitive to riluzole, sorafenib or the combination of both reagents when compared to UACC903 xenografts (FIG. 3C). It was noted that 1205Lu xenografts were more responsive to the combination therapy than UACC903 xenografts in spite of their common B-RAF V600E genotype, indicating that other mutations persistent in these cells can influence their response. Additionally, immunohistochemical analyses were performed on excised xenografts using antibodies against the cleaved form of Caspase 3 to detect apoptotic cell death, and Ki-67 to detect changes in cell proliferation. Single agent riluzole, sorafenib or the combination of both compounds showed a substantial increase in the number of positive Caspase 3 cells in comparison to the controls. Conversely, the number of Ki-67 positive cells was reduced in either single agent or combined treatments. Riluzole had a more potent effect on C8161 and 1205Lu cell lines, despite the disparity in B-RAF status, than UACC903. A combination of riluzole and sorafenib, even though at half the concentration when each agent was used alone, was effective against all three xenografts (FIGS. 3A, 3B and 3C). In vivo xenograft studies were also performed to evaluate the efficacy of riluzole and PLX4720 combination in UACC903 cells. PLX4720 alone was not as potent as riluzole (FIG. 3D). Furthermore, when half the doses of riluzole and PLX4720 were combined, further suppression of tumor progression as observed with similar dosing with the riluzole and sorafenib combination was not detected (compare FIGS. 3B and 3D). Efficacy of the combination of riluzole and PLX4720 against the wild type B-RAF melanoma cell line C8161 was not evaluated with PLX4720 in vivo as it is ineffective in inducing apoptosis in vitro and in vivo, and has also been shown to promote cell growth through activation of the MAPK pathway in a C-RAF dependent manner.

Pre-clinical and clinical trials performed with sorafenib, PLX4720 and riluzole demonstrated a reduction in levels of activated ERK supporting the notion that MAPK is a target for all three compounds. Western immunoblots were performed with protein lysates prepared from in vitro cultured cells or excised in vivo xenografts treated with sorafenib, PLX4720 and riluzole either alone or in combination as described above. Riluzole inhibits the MAPK pathway as measured by a decrease in levels of ERK phosphorylation in a cell line-dependent manner. Sorafenib was found to more highly suppress ERK phosphorylation in UACC903 and 1205Lu cells than in C8161 cells. The combination was, however, capable of suppressing ERK phosphorylation in all three cell lines. PLX4720 was only found to suppress ERK activity in the B-RAF^{V600E} cell line UACC903 as a single agent or in combination, but not in the C8161 cell line. Protein lysates obtained with harvested xenografts showed similar results. The effect of the combination of drugs on the pro-apoptotic protein Mcl-1, which has been shown to be down-regulated by sorafenib, was investigated as a possible target for additive and synergistic inhibition in tumor growth. A reduction in Mcl-1 levels was detected in sorafenib-treated UACC903 and 1205 LU cells while the combination of riluzole and sorafenib led to a reduction in Mcl-1 in all three cells lines. PLX4720, however, does not down regulate the levels of Mcl-1 either by itself or in combination with riluzole.

Riluzole Plus MK2206 Yields Synergistic Suppression in Cell Growth

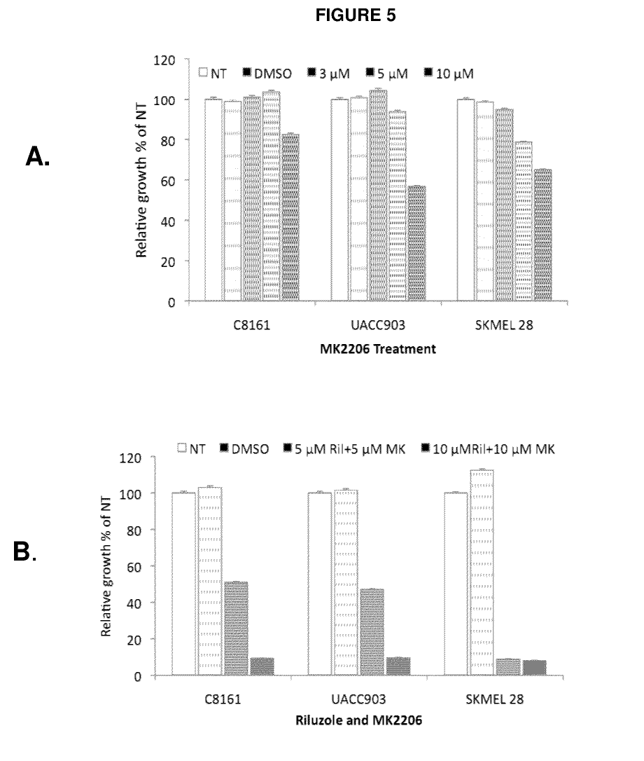

MK2206 was also demonstrated to be highly effective in combination therapy with riluzole. De-regulated PI3K/AKT signaling is a common event in melanoma. In addition, activation of AKT in GRM1-expressing melanoma cells was demonstrated. Cell viability/cell proliferation MTT assays were performed to examine possible inhibition in cell growth by MK2206 in three different melanoma cells line. As a single agent, MK2206 at 10 μM was found to suppress cell proliferation similarly in C8161, UACC903 and SKMEL 28 as 10 μM riluzole (FIG. 5A). However, the efficacy was more pronounced in UACC903 and SKMEL 28 cells, which have mutations in the PTEN tumor suppressor gene compared to C8161, which are PTEN wildtype. However, the combination of riluzole and MK2206 at either 5 μM or 10 μM of each compound was found to have synergistic suppressive activity in the proliferation of each of the cell lines with greater efficacy than either of the compounds alone, with the 10 μM rate of each compound having a more profound effect than 5 μM (FIG. 5B).

Riluzole Plus MK2206 Produces a Reduction in PI3K/AKT Signaling Cascades

To examine whether the enhanced anti-melanoma cell growth activity detected in the combination of riluzole and MK2206 was in part due to a decrease in the levels of activated PI3K/AKT, Western immunoblots were performed and probed with phosphorylated AKT antibody. Riluzole has previously been shown to suppress AKT phosphorylation in melanoma cell lines in vitro. Furthermore, correlations were detected between reduced phospho-AKT and post-riluzole treated biopsy samples from patients that were responsive to riluzole in a Phase 0 clinical trial, suggesting that in in vivo settings a decrease in levels of activated AKT may contribute to reduced tumor progression. It was determined whether a decrease in the number of viable cells in three human melanoma cell lines in the presence of MK2206 or combining with riluzole also reflected a reduction in levels of phospho-AKT. Riluzole and MK2206 both suppress AKT phosphorylation as a single agent, with MK2206 showing a greater effect due to its high specificity for AKT. Furthermore, it was observed that the combination of MK2206 with riluzole increased the suppression in the phosphorylation of AKT in all three cell lines.

Combined Riluzole with MK2206 Yields Significant Reduction in Tumor Progression in In Vivo Xenografts

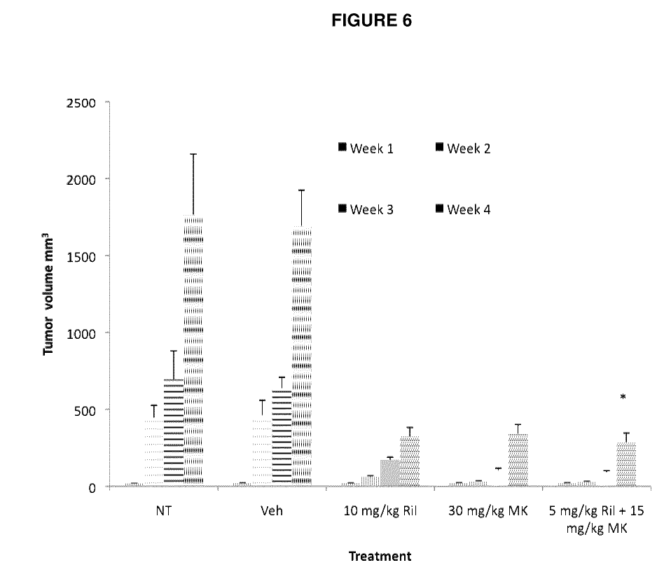

C8161 human melanoma cells were inoculated into the dorsal flanks of immunodeficient nude mice. 10⁶ cells were inoculated into each dorsal flank of 5-6 week-old mice. When the tumors reached tumor volumes of ˜10 mm³, the mice were randomly divided in 5 groups with similar tumor volumes. The mice were then treated by oral gavage daily, except for MK2006 treatment, which was administered three times a week. The mice were given either vehicle (DMSO), Ril—riluzole (10 mg/kg), MK—MK2206 (30 mg/kg) or a combination of the two (5 mg/kg riluzole and 15 mg/kg MK2206), as well as a no treatment (NT) control group. Tumor volumes were determined once a week with a vernier caliper and the experiment was terminated when tumor burden in the control animals reached maximum permitted levels. Riluzole and MK2206 as single agents were found to suppress the growth of the melanoma xenografts (FIG. 6). The combination of both compounds when given at half of the dose of single agents yielded significant reduction in tumor volumes in comparison to no treatment or vehicle-treated group (FIG. 6, p<0.005).

Riluzole with MK2206 Combination Increases Caspase-3 Cleavage and Decreases Ki-67 Expression

Excised tumor samples were subjected to immunohistochemistry evaluation with the apoptosis marker, cleaved Caspase-3, and the proliferation index marker, Ki-67. Quantification of the stained slides for cleaved Caspase-3 showed a significant increase in apoptotic cells when comparing the untreated or vehicle-treated samples with either riluzole (10 mg/kg) or MK2206 alone (30 mg/kg). The combination of riluzole (5 mg/kg) and MK2206 (15 mg/kg) showed an even higher number of cleaved Caspase-3-positive cells, which was statistically significant when compared to riluzole or MK2206 as a single agent (p<0.001). Similarly, staining and quantification for the proliferation marker Ki-67 showed actively proliferating cells in the untreated or vehicle-treated samples. Treatment of these tumor-bearing mice with single agent riluzole or MK2206 led to a decrease in the number of Ki-67 positive cells, with further reductions observed with the combination treatment at half the dose of each compound (p<0.001).

### SUMMARY OF EXAMPLES

Phase 0 and Phase II clinical trials with riluzole, which functions as a putative antagonist of GRM1 signaling, show modest anti-tumor activity as a single agent. As shown above, melanoma cells which harbor the most commonly known mutations in B-RAF, (V600E), were less sensitive to the single agent riluzole in both in vitro MTT cell viability cell proliferation and anchorage independent colony assays. Different combinations of riluzole and other inhibitors of downstream targets were assessed. Sorafenib, a small molecule inhibitor originally identified as a RAF kinase inhibitor that also inhibits several receptor tyrosine kinases involved in tumor progression and tumor angiogenesis, was utilized. Sorafenib is FDA-approved for the treatment of hepatocellular carcinoma, and is also a second line agent in the treatment of renal cell carcinoma. As demonstrated above, the combination of riluzole and sorafenib has an additive or synergistic effect in both B-RAF mutant and B-RAF wild type melanoma cells, both in vitro and in vivo.

In cultured cell studies, sorafenib was not very effective in suppressing C8161 cell growth, while it was effective in reducing the number of viable cells in both UACC903 and 1205Lu melanoma cell lines with mutated B-RAF. The in vitro combinatorial studies in C8161 cells using riluzole and sorafenib showed a synergistic reduction in the number of viable cells, while exerting an additive effect detected in UACC903 and 1205Lu cell lines under similar conditions. These results were also observed in in vivo xenograft studies where the combination of riluzole and sorafenib again led to a considerable reduction in tumor progression, as evidenced by the decrease in tumor volumes over time in all three cell lines compared to controls. Thus, sorafenib enhances the cytotoxic effects of riluzole through suppression of downstream targets of GRM1 signaling, including the MAPK pathway. Further, stimulation of GRM1 was shown to modulate MAPK via the ERK mediated signaling pathway in GRM1-expressing human melanoma cells

In the foregoing pre-clinical study examining the efficacy with the combination of riluzole and sorafenib, a broad kinase inhibitor, additive or synergistic suppression in xenograft tumor progression in the pre-clinical combinatorial approaches was observed.

Combining riluzole with the AKT inhibitor MK2206 resulted in suppressed tumor cell growth in vitro. As a single agent, MK2206 suppressed cell proliferation in vitro and its activity was further enhanced when combined with riluzole (FIG. 5B). Furthermore, MK2206 augmented the ability of riluzole to suppress AKT phosphorylation. These in vitro results were further supported in in vivo xenograft studies with half the dose of each reagent (FIG. 6), and confirmed by immunohistochemical staining of apoptotic or proliferation markers.

What is claimed is:

1. A method of treating melanoma in a mammal in need of such treatment comprising administering to said mammal:
(a) a therapeutically effective amount of an inhibitor of metabotropic glutamate receptor 1 (GRM1); and
(b) a therapeutically effective amount of an inhibitor of at least one downstream signaling target of GRM1.

2. The method of claim 1 wherein said mammal is a human being.

3. The method of claim 1 wherein said inhibitor of GRM1 comprises riluzole.

4. The method of claim 1 wherein said downstream signaling target of GRM1 is selected from the group consisting of AKT, MAPK, PI3K/AKT and a combination of two or more thereof.

5. The method of claim 1 wherein said inhibitor of at least one downstream signaling target of GRM1 is selected from the group consisting of MK2206, sorafenib and a mixture thereof.

6. The method of claim 1 wherein said inhibitor of at least one downstream signaling target of GRM1 comprises MK2206.

7. The method of claim 1 wherein said inhibitor of at least one downstream signaling target of GRM1 comprises sorafenib.

8. The method of claim 1 wherein said inhibitor of GRM1 comprises riluzole and said inhibitor of at least one downstream signaling target of GRM1 comprises MK2206.

9. The method of claim 1 wherein said inhibitor of GRM1 comprises riluzole and said inhibitor of at least one downstream signaling target of GRM1 comprises sorafenib.

10. The method of claim 1 wherein said inhibitor of GRM1 and said inhibitor of at least one downstream signaling target of GRM1 are co-administered.

11. The method of claim 10, wherein said inhibitor of GRM1 and said inhibitor of at least one downstream signaling target of GRM1 are co-administered as a combination.

12. The method of claim 11, wherein said combination is a unitary formulation.

13. The method of claim 1 wherein said inhibitor of GRM1 and said inhibitor of at least one downstream signaling target of GRM1 are administered separately, with a time delay.

14. A composition for treating melanoma comprising:
(a) a therapeutically effective amount of an inhibitor of metabotropic glutamate receptor 1 (GRM1); and
(b) a therapeutically effective amount of an inhibitor of at least one downstream signaling target of GRM1.

15. The composition of claim 14 wherein said inhibitor of GRM1 comprises riluzole.

16. The composition of claim 14 wherein said downstream signaling target of GRM1 is selected from the group consisting of AKT, MAPK, PI3K/AKT and a combination of two or more thereof.

17. The composition of claim 14 wherein said inhibitor of at least one downstream signaling target of GRM1 is selected from the group consisting of MK2206, sorafenib and a mixture thereof.

18. The composition of claim 14 wherein said inhibitor of at least one downstream signaling target of GRM1 comprises MK2206.

19. The composition of claim 14 wherein said inhibitor of at least one downstream signaling target of GRM1 comprises sorafenib.

20. The composition of claim 14 wherein said inhibitor of GRM1 comprises riluzole and said inhibitor of at least one downstream signaling target of GRM1 comprises MK2206.

21. The composition of claim 14 wherein said inhibitor of GRM1 comprises riluzole and said inhibitor of at least one downstream signaling target of GRM1 comprises sorafenib.

22. A kit for the treatment of melanoma comprising a container comprising (a) an inhibitor of GRM1 and a container comprising (b) an inhibitor of at least one downstream signaling target of GRM1.
